(19) United States
(12) Patent Application Publication
(10) Pub. No.: US 2023/0183738 A1
(43) Pub. Date: Jun. 15, 2023

(54) UTILIZATION OF PERICARP COLOR1 (P1) AND OTHER ANTHOCYANIN GENES AS SEED MARKERS FOR WHEAT

(71) Applicant: PIONEER HI-BRED INTERNATIONAL, INC., JOHNSTON, IA (US)

(72) Inventors: ANDREW MARK CIGAN, MADISON, WI (US); MANJIT SINGH, JOHNSTON, IA (US)

(73) Assignee: PIONEER HI-BRED INTERNATIONAL, INC., JOHNSTON, IA (US)

(21) Appl. No.: 18/064,423

(22) Filed: Dec. 12, 2022

Related U.S. Application Data
(60) Provisional application No. 63/265,393, filed on Dec. 14, 2021.

(51) Int. Cl. C12N 15/82; C07K 14/415; A01H 1/02

(52) U.S. Cl. CPC C12N 15/8289; C07K 14/415; C12N 15/8231; A01H 1/023

(57) ABSTRACT

Compositions and methods are provided for screening wheat seed for sorting and selection. Compositions comprise polynucleotides and polypeptides, and fragments and variants thereof, which encode and express a screenable color marker in seeds. Expression cassettes comprise a plant-derived polynucleotide, or fragment or variant thereof, operably linked to a promoter, wherein expression of the polynucleotide modulates the color, opacity, fluorescence, or other property of the seed. The plant-derived marker can be used in a male-sterile production system of hybrid wheat seed. Methods for maintaining a line of male-sterile plants and for restoring male fertility in a male-sterile plant, comprising a screenable color marker are provided.

### CROSS REFERENCE TO RELATED APPLICATIONS

This application claims the benefit of U.S. Provisional Application No. 63/265,393, filed Dec. 14, 2021, the entire contents of which is herein incorporated by reference.

### FIELD OF THE INVENTION

The present disclosure relates generally to the fields of plant molecular biology, genetics and plant breeding, specifically, compositions and methods relating to the use of anthocyanin genes as plant seed markers.

### REFERENCE TO ELECTRONICALLY-SUBMITTED SEQUENCE LISTING

The official copy of the sequence listing is submitted electronically via Patent Center as an XML formatted sequence listing with a file named 7771-US-NP.xml created on Nov. 29, 2022 and having a size of 33,103 bytes and is filed concurrently with the specification. The sequence listing comprised in this XML formatted document is part of the specification and is herein incorporated by reference in its entirety.

### BACKGROUND OF THE INVENTION

Development of hybrid plant breeding has made possible considerable advances in quality and quantity of crops produced. Increased yield and combination of desirable characteristics, such as resistance to disease and insects, heat and drought tolerance, along with variations in plant composition are all possible because of hybridization procedures. These procedures frequently rely heavily on providing for a male parent contributing pollen to a female parent to produce the resulting hybrid.

Field crops are bred through techniques that take advantage of the plant's method of pollination. A plant is self-pollinated if pollen from one flower is transferred to the same or another flower of the same plant or a genetically identical plant. A plant is cross-pollinated if the pollen comes from a flower on a genetically different plant.

In certain species, such as Brassica campestris, the plant is normally self-sterile and can only be cross-pollinated. In predominantly self-pollinating species, such as soybeans, wheat, and cotton, the male and female plants are anatomically juxtaposed such that during natural pollination, the male reproductive organs of a given flower pollinate the female reproductive organs of the same flower.

The development of hybrid cultivars of various plant species depends upon the capability of achieving essentially complete cross-pollination between parents. This is most simply achieved by rendering one of the parent lines male sterile (i.e. bringing them in a condition so that pollen is absent or nonfunctional) either manually, by removing the anthers, or genetically by using, in the one parent, cytoplasmic or nuclear genes that prevent anther and/or pollen development (for a review of the genetics of male sterility in plants see Kaul, Male sterility in higher plants. Vol. 10. Springer Science & Business Media, 2012).

The genetic male sterility approach to male-sterility is when the chromosomal nuclear genes of the plant cause the male-sterility. In every presently known inheritable trait which produces male sterility, the sterility is determined by a single gene, and the allele for male-sterility is recessive. The possibility of using genetic male-sterile lines has long been available to producers of hybrid seed but has not proved sufficiently practical for common use. The difficulty being in maintaining an inbred stock which is homozygous for the recessive allele giving rise to male-sterility. The reason for this is plants carrying the homozygous trait for male-sterility are incapable of producing the pollen necessary to self-pollinate or pollinate siblings also homozygous for the recessive allele. In order to maintain a stock of seeds that give rise to male-sterile plants, it is necessary to cross-pollinate male-sterile plants with male-fertile plants, the progeny of which will give rise to a mix of male-sterile and male-fertile plants.

### BRIEF SUMMARY OF THE INVENTION

Compositions and methods for utilization of maize P1 gene and other anthocyanin genes as plant-based screenable markers in wheat seeds are provided. Compositions include expression cassettes having a polynucleotide encoding a plant-based screenable marker for seed selection, or fragments or variants thereof, operably linked to a promoter that expresses in seed, wherein expression of the polynucleotide modulates the color or opacity or other property of the seed of the plant. Compositions may also comprise regulatory elements, including but not limited to, enhancer elements and introns to enhance the expression of these polynucleotides. Also provided are compositions comprising expression cassettes comprising one or more male-fertility restoration polynucleotides, or fragments or variants thereof, operably linked to a polynucleotide encoding a screenable marker for seed selection, which is operably linked to a promoter that expresses in seed, wherein expression of the one or more male-fertility restoration polynucleotides modulates the male fertility of a plant and the expression of the polynucleotide encoding a plant-based screenable marker modulates the color or opacity or other property of the seed of the plant. Various methods are provided for increasing seed from a plant, where the seed can be sorted based on the expression of screenable marker. Methods for identifying and/or selecting wheat seeds that are homozygous for one or more mutations that confer nuclear recessive male sterility and/or seeds that contain male-fertility restoration polynucleotides operably linked to a polynucleotide encoding a screenable marker are also provided.

### SEQUENCE LISTING

Nucleic acid and protein sequences listed in the accompanying sequence listing and referenced herein are shown using standard letter abbreviations for nucleotide bases and amino acids. Only one strand of each nucleic acid sequence is shown, but the complementary strand is understood to be included by any reference to the displayed strand. Sequence listings are described in the following Table 1.

TABLE 1

| SEQ ID NO: | Name | Description |
|---|---|---|
| 1 | ZM-P1 WT | Z. mays P1 genomic sequence |
| 2 | ZM-P1 WT | Z. mays P1 protein sequence |
| 3 | ZM-P1 TRUNC | Z. mays P1 genomic sequence truncated |
| 4 | ZM-P1 TRUNC | Z. mays P1 protein sequence truncated |
| 5 | TA-P1-4A | T. aestivum P1 genomic sequence Chrom 4A |
| 6 | TA-P1-4A | T. aestivum P1 protein sequence Chrom 4A |
| 7 | TA-P1-1D | T. aestivum P1 genomic sequence Chrom 1D |
| 8 | TA-P1-1D | T. aestivum P1 protein sequence Chrom 1D |
| 9 | OS-KALA4 | O. sativa KALA4 genomic sequence |
| 10 | OS-KALA4 | O. sativa KALA4 protein sequence |
| 11 | alpha amylase | Z. mays alpha amylase genomic sequence |
| 12 | alpha amylase | Z. mays alpha amylase protein sequence |
| 13 | CAMV 35S enhancer | cauliflower mosaic virus 35S enhancer |
| 14 | LTP2 promoter | barley lipid transfer protein promoter |
| 15 | PG47 promoter | Z. mays PG47 promoter |
| 16 | CZ19B1 promoter | maize 19KD B1 Zein gene CZ19B1 promoter |
| | | maize 27 KD Gamma zein gene GZ-W64A |
| 17 | GZ-W64A promoter | promoter |
| 18 | ZM-SH1-INT | tron of maize shrunken 1 sucrose synthase gene |

### DETAILED DESCRIPTION

All publications and patent applications mentioned in the specification are indicative of the level of those skilled in the art to which this invention pertains. All publications and patent applications are herein incorporated by reference to the same extent as if each individual publication or patent application was specifically and individually indicated to be incorporated by reference.

The maize P1 (Pericarp color1) gene regulates the phlobaphene biosynthesis pathway and imparts color to the seed pericarp and other parts of the plant. Described herein are methods and compositions for utilization of polynucleotides encoding Pericarp color1 (P1) polypeptides and other anthocyanin genes as screenable seed markers in wheat or other plants. In some examples, the polynucleotide encoding the screenable marker is expressed in a seed, for example, in the endosperm, aleurone, cotyledon, embryo, or seed coat, or combinations thereof. Accordingly, in some embodiments, the polynucleotide encoding the screenable marker is operably linked to a heterologous promoter that expresses in seed.

In some embodiments, the polynucleotide encoding the screenable marker includes a nucleotide sequence of SEQ ID NO:1, 3, 5, 7, or 9, its variants, or fragments thereof; a nucleotide sequence that is at least 80%, at least 85%, at least 90%, at least 95%, at least 96%, at least 97%, at least 98%, or at least 99% identical to the nucleotide sequence of SEQ ID NO: 1, 3, 5, 7, or 9, its variants, or fragments thereof; a nucleotide that encodes a polypeptide with an amino acid sequence of SEQ ID NO: 2, 4, 6, 8, or 10; or a nucleotide that encodes a polypeptide that is at least 85%, 90%, 91%, 92%, 93%, 94%, 95%, 96%, 97%, 98%, or 99% identical to the amino acid sequence of SEQ ID NO: 2, 4, 6, 8, or 10. In some aspects, the percent identity is determined with respect to the full length nucleotide sequence of SEQ ID NO: 1, 3, 5, 7, or 9 or the full length amino acid sequence of SEQ ID NO: 2, 4, 6, 8, or 10. As used herein, the term “fragment” refers to a portion of a nucleotide sequence and hence the protein encoded thereby or a portion of an amino acid sequence. Fragments of a nucleotide sequence may encode protein fragments that retain the biological activity of the native protein. As shown herein, it was found that the maize P1 gene could be considerably shortened while still retaining its capability of conditioning anthocyanin production in the seeds of wheat plants. Examples of a shortened maize P1 gene sequence for instance is SEQ ID NO:3 and its protein sequence, SEQ ID NO:4. Accordingly, in some aspects, the fragments encoding the screenable marker comprises at least 200, 300, or 400 contiguous amino acids of the polypeptides of SEQ ID NO: 2, 4, 6, 8, or 10. In some aspects, the polynucleotide encoding the screenable marker operably linked to a heterologous promoter that expresses in seed is included in a recombinant DNA construct.

In some embodiments, the polynucleotide is operably linked to a heterologous promoter that functions in a plant cell, wherein the heterologous promoter is an inducible promoter, a constitutive promoter, or a tissue-specific or preferred promoter.

As used herein, “promoter” includes reference to a regulatory region of DNA usually comprising a TATA box or a DNA sequence capable of directing RNA polymerase II to initiate RNA synthesis at the appropriate transcription initiation site for a particular coding sequence. A promoter may additionally comprise other recognition sequences generally positioned upstream or 5′ to the TATA box or the DNA sequence capable of directing RNA polymerase II to initiate RNA synthesis, referred to as upstream promoter elements, which influence the transcription initiation rate. The promoter may be native or homologous or foreign or heterologous to the host or could be the natural sequence or a synthetic sequence.

A “plant promoter” is a promoter capable of initiating transcription in plant cells. Exemplary plant promoters include, but are not limited to, those that are obtained from plants, plant viruses, and bacteria which comprise genes expressed in plant cells such as from Agrobacterium or Rhizobium. Examples of promoters under developmental control include promoters that preferentially initiate transcription in certain tissues, such as leaves, roots, seeds, fibers, xylem vessels, tracheids, or sclerenchyma. Such promoters are referred to as “tissue preferred.” Promoters that initiate transcription only in certain tissues are referred to as “tissue specific.” A “cell type” specific promoter primarily drives expression in certain cell types in one or more organs, for example, vascular cells in roots or leaves. An “inducible” promoter is a promoter that is under environmental control. Examples of environmental conditions that may affect transcription by inducible promoters include anaerobic conditions or the presence of light. Tissue specific, tissue preferred, cell-type specific, and inducible promoters constitute the class of “non-constitutive” promoters.

In some examples, the polynucleotide is operably linked to a promoter that expresses in a seed, including but not limited to a seed-specific or seed-preferred promoter. The promoter may optionally include a regulatory element, such as an enhancer element or intron.

Any suitable promoter that expresses in seeds may be used. In one aspect, a promoter that directs expression to particular tissue within the seed may be desirable, such as endosperm or aleurone. A promoter that directs expression to a particular tissue refers includes tissue-specific and tissue-preferred promoters. In some aspects, suitable promoters include those that express highly in the plant seed, express more in the plant seed tissue than in other plant tissue, or express exclusively in the plant seed tissue.

For example, “seed-specific” promoters may be employed to drive expression of the screenable marker. Specific seed promoters include those promoters active during seed development, promoters active during seed germination, and/or that are expressed only in the seed. Seed-specific promoters, such as annexin, P34, β-phaseolin, α subunit of β-conglycinin, oleosin, zein, napin promoters have been identified in many plant species such as maize, wheat, rice and barley. See U.S. Pat. Nos. 7,157,629, 7,129,089, and 7,109,392. Such seed-preferred promoters further include, but are not limited to, Cim1 (cytokinin-induced message); cZ19B1 (maize 19 KD zein); and milps (myo-inositol-1-phosphate synthase); (see WO 00/11177, herein incorporated by reference).

Seed-specific promoters also include those that express in endosperm and/or embryo. One example of an endosperm-specific promoter is the 27 KD gamma-zein promoter. The maize globulin-1 and oleosin promoters are examples of embryo-specific promoters. For dicots, other seed-specific promoters include, but are not limited to, bean β-phaseolin, napin, β-conglycinin, soybean lectin, cruciferin, and the like. For monocots, seed-specific promoters include, but are not limited to, promoters of the 15 KD beta-zein, 22 KD alpha-zein, 27 KD gamma-zein, waxy, shrunken 1, shrunken 2, globulin 1, an LTP1, an LTP2, and oleosin genes.

Seed-preferred promoters include those that express preferentially in seed. See, for example, WO 00/12733, where seed-preferred promoters from end1 and end2 genes are disclosed. WO 00/12733 is herein incorporated by reference in its entirety.

The polynucleotides encoding the screenable markers may also include enhancers, either translation or transcription enhancers, as may be required. These enhancer regions are well known to persons skilled in the art and can include the ATG initiation codon and adjacent sequences. The initiation codon must be in phase with the reading frame of the coding sequence to ensure translation of the entire sequence. The translation control signals and initiation codons can be from a variety of origins, both natural and synthetic. Translational initiation regions may be provided from the source of the transcriptional initiation region, or from the structural gene. The sequence can also be derived from the regulatory element selected to express the gene and can be specifically modified to increase translation of the mRNA. It is recognized that to increase transcription levels enhancers may be utilized in combination with promoter regions. It is recognized that to increase transcription levels, enhancers may be utilized in combination with promoter regions. Enhancers are nucleotide sequences that act to increase the expression of a promoter region. Enhancers are known in the art and include the SV40 enhancer region, the 35S enhancer element and the like. Some enhancers are also known to alter normal promoter expression patterns, for example, by causing a promoter to be expressed constitutively when without the enhancer, the same promoter is expressed only in one specific tissue or a few specific tissues.

In some examples, the coding region of a screenable color marker gene, preferably the P1 gene, is operably linked to a promoter that directs expression in at least in the seed, including but not limited to the promoters of the barley lipid transfer protein LTP2 gene, the maize 19 KD B1 Zein CZ19B1 gene, or the maize 27 KD Gamma zein GZ-W64A gene. In some aspects, the polynucleotide is full length or truncated, such as those set forth in SEQ ID NO:1, SEQ ID NO:5 or SEQ ID NO:3.

As desired, the polynucleotides that encode the screenable markers may be modified to increase its expression in the plant, for example, to increase the expression of the screenable marker in a plant, plant part thereof, or seed. In some aspects, the regulatory region of the polynucleotide may be modified to increase expression of the screenable marker, for example, by editing the existing regulatory region to replace, delete, and/or insert nucleotides for improved expression, for example to include an enhancer element or an intron. See, for example, PCT patent publication WO2018183878, published Oct. 4, 2018, incorporated herein by reference in its entirety.

As shown in Example 1, the expression of maize P1 full length gene (SEQ ID NO:1) and P1-truncated (trunc) gene (SEQ ID NO:3) in wheat seed were tested using various seed-specific promoters and enhancer elements for their ability to impart color to the wheat seeds. Such promoters and enhancer elements include, but are not limited to, cauliflower mosaic virus (CaMV) 35S enhancer, barley lipid transfer protein (LTP2) promoter, maize 19KD B1 Zein gene CZ promoter, maize 27 KD Gamma zein gene GZ-W64A promoter, or the intron of maize shrunken 1 sucrose synthase gene, Zm-SH1-INT.

In some examples, a LTP2 promoter with or without an enhancer, such as CAMV 35S, is used drive the expression of the polynucleotide encoding the screenable marker, including but not limited to the Zm-P1-trunc sequence, in seed. In some examples, a LTP2 promoter with or without an intron, such as Zm-SH1-INT, is used drive the expression of Zm-P1-trunc sequence, in seed. In some examples, maize 19KD B1 Zein gene CZ promoter with or without an enhancer, such as CAMV 35S, is used drive the expression of Zm-P1-trunc sequence, in seed. In some examples, the maize 27 KD Gamma zein gene GZ-W64A promoter with or without an enhancer, such as CAMV 35S, is used drive the expression of Zm-P1-trunc sequence, in seed.

As shown in Example 2, the expression of maize P1 full length gene (SEQ ID NO:1) and P1-truncated (trunc) gene (SEQ ID NO:3) in wheat seed were tested using various seed-specific promoters and enhancer elements for their ability to impart color to the wheat seeds. Such promoters and enhancer elements included cauliflower mosaic virus (CaMV) 35S enhancer, barley lipid transfer protein (LTP2) promoter, maize 19KD B1 Zein gene CZ promoter, maize 27 KD Gamma zein gene GZ-W64A promoter, or the intron of maize shrunken 1 sucrose synthase gene, Zm-SH1-INT.

As shown in Example 3, the expression of wheat Ta-P1-4A protein from the homolog group chromosome 4 and wheat Ta-P1-1D protein from homolog group chromosome 1 in wheat seed were tested using various seed-specific promoters and enhancer elements for their ability to impart color to wheat seeds. Such promoters and enhancer elements included cauliflower mosaic virus (CaMV) 35S barley lipid transfer protein (LTP2) promoter, maize 19KD B1 Zein gene CZ19B1 promoter, maize 27 KD Gamma zein gene GZ-W64A promoter, or the intron of maize shrunken 1 sucrose synthase gene, Zm-SH1-INT. In some embodiments, a fused CAMV 35S enhancer and LTP2 promoter, were used to drive the expression of Ta-P1-4A and Ta-P1-1D.

The expression of the native wheat Ta-P1-4A gene may also be modulated, possibly through CRISPR-mediated genome editing, to create a maintainer chromosome. In an aspect, the native promoter of Ta-P1-4A gene is swapped with the promoter of an endosperm-specific wheat gene, or alternatively an appropriate expression enhancing element is inserted into the promoter of Ta-P1-4A that can render seed specificity. In a further aspect, an endosperm-specific promoter such as Zea mays LTP2 promoter is inserted before the native wheat P1 gene, or the native wheat P1 promoter is replaced with Zm-LTP2 promoter (promoter swap). This manipulation generates a maintainer chromosome which may be combined with a suitable mutation in any of the linked male fertility genes. In an aspect, seeds from such a maintainer chromosome segregate 3:1 for colored and non-colored seeds, and non-colored seeds will generate male sterile plants.

The guide RNA/Cas endonuclease system described herein can be used to allow for the insertion or deletion of a promoter element from either a transgenic (pre-existing, artificial) or endogenous gene. In an aspect, promoter elements, such as enhancer elements, are introduced in promoters driving gene expression cassettes in multiple copies (e.g., 3×=3 copies of enhancer element) for trait gene testing or to produce transgenic plants expressing specific trait. Enhancer elements include, but are not limited to, SV40 enhancer region and the 35S enhancer element. In some events, the enhancer elements can cause an unwanted phenotype, a yield drag, or a change in expression pattern of the trait of interest that is not desired. Consequently, it may be desired to insert or remove extra copies of the enhancer element while keeping the trait gene cassettes intact at their integrated genomic location. In an aspect, the guide RNA/Cas endonuclease system described herein is used to insert a desired enhancing element or to remove an unwanted enhancing element from the plant genome. In a further aspect, the guide RNA is designed to contain a variable targeting region targeting a target site sequence of 12-30 bps adjacent to a NGG (PAM) in the enhancer. The Cas endonuclease cleaves to insert or remove one or multiple enhancers. In a further aspect, the guideRNA/Cas endonuclease system is introduced by either Agrobacterium or particle gun bombardment. Alternatively, two different guide RNAs (targeting two different genomic target sites) can be used to insert or remove one or more enhancer elements into or from the genome of an organism, in a manner similar to the insertion or removal of a (transgenic or endogenous) promoter described herein.

One of the most important characteristics of the maintainer chromosome is the lack of recombination between the male fertility gene and the color marker gene, Ta-P1-4A. While the most tightly linked gene to Ta-P1-4A is TaMs9 with a distance of 7.7 Mb between them, Ms45 and Ms26 genes are more distantly placed compared to Ta-P1-4A and therefore are less tightly linked. To effectively utilize Ms45 and Ms26 genes to create maintainer chromosome, or to further tighten the linkage between Ms9 and Ta-P1-4A, the distance between the fertility genes and Ta-P1-4A is reduced to create a tighter linkage. In an aspect, native physical mutagenesis techniques such as Gamma radiations or genome editing techniques (e.g., CRISPR-Cas) are used to reduce the physical distance of fertility genes, including but not limited to Ms9, Ms26, and Ms45, and Ta-P1-4a on the same chromosome, thereby creating tighter linkage.

The utilization of Ta-P1-4A as a marker to maintain male sterile inbreds, as outlined in Example 3, can be further expanded to specifically place the Ms1-P1 male sterility/marker gene TDNA on a chromosome from an alien species, including the 4E, 4EL, or 4H chromosome from Thinopyrum, Aegilops, Secale, Haynaldia, Elyymus, or Hordeum for example, that has been introduced into wheat through traditional breeding. Such modification does not alter the genomic composition of wheat chromosomes but provides the benefits of the maintainer system as outlined herein. The addition of an extra chromosome in wheat results in creation of a monosomic addition line. The term “monosomic” means that one chromosome of a homologous pair is missing, while the term “disomic” means that both chromosomes of a homologous pair are present. Hexaploid wheat has 42 chromosomes, so a monosomic wheat plant has 2n−1=41 chromosomes. Monosomics segregate in a non-Mendelian pattern. Monosomic wheat plants produce ˜75% nullisomic (n−1=20) female gametes. However, the monosomics do produce disomics at some frequency, which can fix the maintainer genotype. The added advantage of this system would be the elimination of the production of disomics.

As shown in Example 8, the expression of rice Kala4 protein in wheat seed were tested using various seed-specific promoters and enhancer elements for their ability to impart color to wheat seeds. Such promoters and enhancer elements may include, but are not limited to, cauliflower mosaic virus (CaMV) 35S barley lipid transfer protein (LTP2) promoter, maize 19KD B1 Zein gene CZ19B1 promoter, maize 27 KD Gamma zein gene GZ-W64A promoter, or the intron of maize shrunken 1 sucrose synthase gene, Zm-SH1-INT. In certain aspects, a recombinant DNA construct comprising a LTP2 promoter was transcriptionally fused to the rice Kala4 genomic sequence, excluding the Kala4 promoter, to drive expression.

A method of identifying seeds comprising a screenable marker is provided, wherein the method includes identifying seeds that comprise a plant-derived polynucleotide encoding a screenable marker operably linked to a promoter that expresses in seed. In some aspects, the polynucleotide encoding the screenable marker includes a nucleotide sequence of SEQ ID NO:1, 3, 5, 7, or 9, its variants, or fragments thereof; a nucleotide sequence that is at least 80%, at least 85%, at least 90%, at least 95%, at least 96%, at least 97%, at least 98%, or at least 99% sequence identical to the nucleotide sequence of SEQ ID NO: 1, 3, 5, 7, or 9, its variants, or fragments thereof; a nucleotide that encodes a polypeptide with an amino acid sequence of SEQ ID NO: 2, 4, 6, 8, or 10; or a nucleotide that encodes a polypeptide that is at least 85%, 90%, 91%, 92%, 93%, 94%, 95%, 96%, 97%, 98%, or 99% identical to the amino acid sequence of SEQ ID NO: 2, 4, 6, 8, or 10. In some aspects, the percent identity is determined with respect to the full length nucleotide sequence of SEQ ID NO: 1, 3, 5, 7, or 9 or the full length amino acid sequence of SEQ ID NO: 2, 4, 6, 8, or 10. In some aspects, the nucleic acid fragments encode a screenable marker that comprises at least 200, 300, or 400 contiguous amino acids of the polypeptides of SEQ ID NO: 2, 4, 6, 8, or 10. In some aspects, the polynucleotide encoding the screenable marker operably linked to a heterologous promoter that expresses in seed is included in a recombinant DNA construct.

In a further aspect of the method, the seeds are identified based on the expression of the screenable marker, wherein the expression of the screenable marker results in a change in seed color, seed opacity, or seed property, such as fluorescence, as compared to seed not comprising the polynucleotide encoding the screenable marker operably linked to the promoter that expresses in seed.

The utilization of such a plant-based screenable marker may be used to facilitate the assembly of a system for production of hybrid wheat seed and alternatively, or addition to, may be used for trait discovery purposes where markers are required.

Methods of increasing seeds are provided herein. In an aspect, a method of increasing seeds includes crossing male parent wheat plants to fertilize male-sterile female wheat parent plants to produce seeds, where the male-sterile female parent wheat plants comprise one or more homozygous mutations of a male-fertility polynucleotide that confers male-sterility to the plant, where the male parent wheat plants comprise one or more male-fertility restoration polynucleotides that functionally complements the male-sterility phenotype in the male-sterile wheat plant, and where the one or more male-fertility restoration polynucleotides is operably linked to a plant-derived polynucleotide encoding a screenable marker for seed selection, where the polynucleotide encoding the screenable marker is operably linked to a promoter that expresses in seed.

In some examples, the polynucleotide that encodes the screenable marker is endogenous or native with respect to the male-fertility restoration polynucleotides. As used herein, the term “endogenous” or “native” or “natively” means normally present in the specified plant, present in its normal state or location in the chromosome (non-modified), plant cell, or plant. In some embodiments, the polynucleotide that encodes the screenable marker, such as wheat P1 polynucleotides, is endogenous or native with respect to the wheat male-fertility restoration Ms9, Ms26 and Ms45 polynucleotides in wheat.

In another aspect, a method of increasing seeds includes self-fertilizing a wheat plant to produce seeds, where the wheat plant comprises one or more homozygous mutations of a male-fertility polynucleotide that confers male-sterility to the plant, and one or more male-fertility restoration polynucleotides that functionally complements the male-sterility phenotype in the male-sterile wheat plant, and where the one or more male-fertility restoration polynucleotides is operably linked to a plant-derived polynucleotide encoding a screenable marker for seed selection, where the polynucleotide encoding the screenable marker is operably linked to a promoter that expresses in seed.

The polynucleotide encoding the screenable marker may include a nucleotide sequence of SEQ ID NO:1, 3, 5, 7, or 9, its variants, or fragments thereof; a nucleotide sequence that is at least 80%, at least 85%, at least 90%, at least 95%, at least 96%, at least 97%, at least 98%, or at least 99% sequence identical to the nucleotide sequence of SEQ ID NO: 1, 3, 5, 7, or 9, its variants, or fragments thereof; a nucleotide that encodes a polypeptide with an amino acid sequence of SEQ ID NO: 2, 4, 6, 8, or 10; or a nucleotide that encodes a polypeptide that is at least 85%, 90%, 91%, 92%, 93%, 94%, 95%, 96%, 97%, 98%, or 99% identical to the amino acid sequence of SEQ ID NO: 2, 4, 6, 8, or 10. In some aspects, the percent identity is determined with respect to the full length nucleotide sequence of SEQ ID NO: 1, 3, 5, 7, or 9 or the full length amino acid sequence of SEQ ID NO: 2, 4, 6, 8, or 10. In some aspects, the nucleic acid fragments encode a screenable marker that comprises at least 200, 300, or 400 contiguous amino acids of the polypeptides of SEQ ID NO: 2, 4, 6, 8, or 10. In some aspects, the polynucleotide encoding the screenable marker operably linked to a heterologous promoter that expresses in seed is included in a recombinant DNA construct.

In a further aspect of the method, the male-sterile female parent plants may have one or more homozygous mutations of an endogenous male-fertility polynucleotide so that the mutation(s) confers male-sterility to the plant. As used herein, the term “male-fertility polynucleotide” means one of the polynucleotides critical to a specific step in microsporogenesis, the term applied to the entire process of pollen formation. In some examples, the one or more male-fertility polynucleotides include but are not limited to Ms1, Ms5, Ms9, Ms22, Ms26, or Ms45.

In one aspect, the one or more male-fertility restoration polynucleotides is a Ms1, Ms5, Ms22, Ms26, or Ms45 male-fertility polynucleotide.

The method may also include the step of obtaining a mixture of seeds comprising seeds that will give rise to male-sterile female plants as indicated by the absence of the expression of the screenable marker in seed and seed that will give rise to male-fertile plants as indicated by the presence of the expression of the screenable marker in seed.

In a further aspect of the method, the expression of the screenable marker results in a change in seed color, seed opacity, or other seed property as compared to seed not comprising the polynucleotide encoding the screenable marker operably linked to a promoter that expresses in seed.

Expression of the plant screenable marker in seed allows for the seed to be identified, selected, and/or sorted from seeds that do not contain the male-fertility plant restoration polynucleotides, i.e., those seed that do not have the polynucleotide encoding the screenable marker driven by a promoter that expresses in seed.

The screenable marker may relate to the color, physiology, or morphology of the plant or seed. Examples of seed phenotypes that are suitable markers include but are not limed to seed color, seed color intensity or pattern, fluorescence, seed shape, seed surface texture, seed size including seed size width and/or length, seed density, or other seed characteristics. Examples of seed screenable color markers include but are not limited to Percarp 1 (P1) genes and polynucleotides and Kala4 genes and polynucleotides that have been modified so that they may be expressed in seed. In some examples, the plant-derived polynucleotide is a P1 gene, polynucleotide, or variations thereof and confers a darker color phenotype to the seed. In some embodiments, the plant-derived polynucleotides are polynucleotides encoding P1 color marker that confers a darker color phenotype to the seed when compared to wildtype seed and may be used for seed identification, selection, and sorting. The plant-derived polynucleotide encoding a screenable marker for seed selection may be synthesized, isolated, or obtained from any number of sources, including monocot plants, including but not limited to Zea mays, Triticum, Triticum aestivum, Oryza sativa, and related species.

Seeds may be sorted into various populations using any of the screenable markers described herein that are driven by a promoter that expresses in seed. For example, the absence of the plant screenable marker in the seed, e.g., seed lacking the male-fertility restoration polynucleotides, indicates the seed, when planted, will give rise to a male-sterile female plant. Plants from this seed may be used as male-sterile female inbreds for hybrid and seed increase production. The presence of the plant screenable marker in the seed, e.g. seed having the one or more male-fertility restoration polynucleotides, indicates that the seed will give rise to a male-fertile plant that may be used as a maintainer for the male-sterile female plant. The seeds may be sorted using any suitable approach or instrument so long as it has sufficient sensitivity to detect the difference between screenable marker expressing and non-expressing seeds. The seeds may be manually, mechanically, or optically sorted into these populations using any suitable instrument. To facilitate high throughput and analysis, the sorting may employ a semi-automated or automated approach. Populations of seeds may be sorted using any suitable technology, including but not limited to optical sensing technology such as multi-spectral or hyperspectral imaging, UV, visible or NIR spectroscopy systems, and/or optical scanning.

Methods of restoring male fertility in a male-sterile plant are provided herein. In an aspect of the invention, a method includes introducing into a male-sterile plant, where the male-sterile plant comprises one or more homozygous mutations of a male-fertility polynucleotide that confers male sterility to the plant, one or more male-fertility restoration polynucleotides operably linked to a plant-derived polynucleotide encoding a screenable marker operably linked to a promoter that expresses in seed. In some aspects, the polynucleotide encoding the screenable marker includes a nucleotide sequence of SEQ ID NO:1, 3, 5, 7, or 9, its variants, or fragments thereof; a nucleotide sequence that is at least 80%, at least 85%, at least 90%, at least 95%, at least 96%, at least 97%, at least 98%, or at least 99% sequence identical to the nucleotide sequence of SEQ ID NO: 1, 3, 5, 7, or 9, its variants, or fragments thereof; a nucleotide that encodes a polypeptide with an amino acid sequence of SEQ ID NO: 2, 4, 6, 8, or 10; or a nucleotide that encodes a polypeptide that is at least 85%, 90%, 91%, 92%, 93%, 94%, 95%, 96%, 97%, 98%, or 99% identical to the amino acid sequence of SEQ ID NO: 2, 4, 6, 8, or 10. In some aspects, the percent identity is determined with respect to the full length nucleotide sequence of SEQ ID NO: 1, 3, 5, 7, or 9 or the full length amino acid sequence of SEQ ID NO: 2, 4, 6, 8, or 10. In some aspects, the nucleic acid fragments encode a screenable marker that comprises at least 200, 300, or 400 contiguous amino acids of the polypeptides of SEQ ID NO: 2, 4, 6, 8, or 10. In some aspects, the polynucleotide encoding the screenable marker operably linked to a heterologous promoter that expresses in seed is included in a recombinant DNA construct.

The male-fertility polynucleotides when expressed in the male-sterile plant functionally complements the male-sterility phenotype caused by the one or more mutations in the endogenous male-fertility polynucleotide in the male-sterile plant so that the male-sterile plant becomes male-fertile.

In a further aspect of the method, the male-sterile female parent plants may have one or more homozygous mutations of an endogenous male-fertility polynucleotide so that it confers male-sterility to the plant. The endogenous male-fertility polynucleotide may be a Ms1, Ms5, Ms22, Ms26, or Ms45 male-fertility polynucleotide. In a yet a further aspect of the method, the one or more male-fertility restoration polynucleotides may be a Ms1, Ms5, Ms9, Ms22, Ms26, or Ms45 male-fertility polynucleotide.

In Example 3, a hybrid wheat maintainer comprising a recombinant DNA construct with a P1-trunc (SEQ ID NO:3), alpha amylase (SEQ ID NO:11) and Ms1 polynucleotides was utilized in combination with ms1d mutations (Tucker et al., 2017, Nature Communications, 8: 869) for use in a hybrid wheat seed production system. In one embodiment, a recombinant DNA construct comprising a P1-trunc polynucleotide transcriptionally fused to CAMV 35S enhancer (SEQ ID NO:13) and LTP2 promoter (SEQ ID NO:14) was operably linked to an alpha amylase polynucleotide (SEQ ID NO:11) transcriptionally fused to the maize PG47 promoter (SEQ ID NO:15), and operably linked to a Ms1 genomic fragment which comprised the native promoter and terminator fragment.

### Additional Terms

As used in this application, including the claims, terms in the singular and tie singular forms, “a,” “an,” and “the,” for example, include plural referents, unless the content clearly dictates otherwise. Thus, for example, a reference to “plant,” “the plant,” or “a plant” also refers to a plurality of plants. Furthermore, depending on the context, use of the term, “plant,” may also refer to genetically similar or identical progeny of that plant. Similarly, the term, “nucleic acid,” may refer to many copies of a nucleic acid molecule. Likewise, the term, “probe,” may refer to many similar or identical probe molecules.

Numeric ranges are inclusive of the numbers defining the range, and expressly include each integer and non-integer fraction within the defined range. Unless defined otherwise, all technical and scientific terms used herein have the same meaning as commonly understood by one of ordinary skill in the art.

In order to facilitate review of the various embodiments described in this disclosure, the following explanation of specific terms is provided.

As used herein, the term “wheat” refers to any species of the genus Triticum, including progenitors thereof, as well as progeny thereof produced by crosses with other species. Wheat includes “hexaploid wheat” which has genome organization of AABBDD, comprised of 42 chromosomes, and “tetraploid wheat” which has genome organization of AABB, comprised of 28 chromosomes. Hexaploid wheat includes T. aestivum, T. spelta, T. mocha, T. compactum, T. sphaerococcum, T. vavilovii, and interspecies cross thereof. Tetraploid wheat includes T. durum (also referred to as durum wheat or Triticum turgidum ssp. durum), T. dicoccoides, T. dicoccum, T. polonicum, and interspecies cross thereof. In addition, the term “wheat” includes possible progenitors of hexaploid or tetraploid Triticum sp. such as T. uartu, T. monococcum or T. boeoticum for the A genome, Aegilops speltoides for the B genome, and T. tauschii (also known as Aegilops squarrosa or Aegilops tauschii) for the D genome. A wheat cultivar for use in the present disclosure may belong to, but is not limited to, any of the above-listed species. Also encompassed are plants that are produced by conventional techniques using Triticum sp. as a parent in a sexual cross with a non-Triticum species, such as rye (Secale cereale), including but not limited to Triticale. In some aspects, the wheat plant is suitable for commercial production of grain, such as commercial varieties of hexaploid wheat or durum wheat, having suitable agronomic characteristics which are known to those skilled in the art.

The disclosure encompasses isolated or substantially purified nucleic acid compositions. An “isolated” or “purified” nucleic acid molecule or protein or a biologically active portion thereof is substantially free of other cellular material or components that normally accompany or interact with the nucleic acid molecule or protein as found in its naturally occurring environment or is substantially free of culture medium when produced by recombinant techniques or substantially free of chemical precursors or other chemicals when chemically synthesized. An “isolated” nucleic acid is substantially free of sequences (including protein encoding sequences) that naturally flank the nucleic acid (i.e., sequences located at the 5′ and 3′ ends of the nucleic acid) in the genomic DNA of the organism from which the nucleic acid is derived. For example, in various aspects, an isolated nucleic acid molecule can contain less than about 5 kb, 4 kb, 3 kb, 2 kb, 1 kb, 0.5 kb or 0.1 kb of nucleotide sequences that naturally flank the nucleic acid molecule in genomic DNA of the cell from which the nucleic acid is derived. A protein that is substantially free of cellular material includes preparations of protein having less than about 30%, 20%, 10%, 5%, or 1% (by dry weight) of contaminating protein.

As used herein, the term “variants” is means sequences having substantial similarity with a sequence disclosed herein. A variant comprises a deletion and/or addition of one or more nucleotides or peptides at one or more internal sites within the native polynucleotide or polypeptide and/or a substitution of one or more nucleotides or peptides at one or more sites in the native polynucleotide or polypeptide. As used herein, a “native” nucleotide or peptide sequence comprises a naturally occurring nucleotide or peptide sequence, respectively. For nucleotide sequences, naturally occurring variants can be identified with the use of well-known molecular biology techniques, such as, for example, with polymerase chain reaction (PCR) and hybridization techniques as outlined herein. A biologically active variant of a protein may differ from that native protein by as few as 1-15 amino acid residues, as few as 1-10, such as 6-10, as few as 5, as few as 4, 3, 2, or even 1 amino acid residue.

Variant nucleotide sequences also include synthetically derived nucleotide sequences, such as those generated, for example, by using site-directed mutagenesis. Generally, variants of a nucleotide sequence disclosed herein will have at least 40%, 50%, 60%, 65%, 70%, 75%, 80%, 85%, 90%, 91%, 92%, 93%, 94%, to 95%, 96%, 97%, 98%, 99% or more sequence identity to that nucleotide sequence as determined by sequence alignment programs described elsewhere herein using default parameters. Biologically active variants of a nucleotide sequence disclosed herein are also encompassed. Biological activity may be measured by using techniques such as Northern blot analysis, reporter activity measurements taken from transcriptional fusions, and the like.

Methods for mutagenesis and nucleotide sequence alterations are well known in the art. See, for example, Kunkel, (1985) Proc. Natl. Acad. Sci. USA 82:488-492; Kunkel, et al., (1987) Methods in Enzymol. 154:367-382; U.S. Pat. No. 4,873,192; Walker and Gaastra, eds. (1983) Techniques in Molecular Biology (MacMillan Publishing Company, New York) and the references cited therein, herein incorporated by reference in their entirety. Guidance as to appropriate amino acid substitutions that do not affect biological activity of the protein of interest may be found in the model of Dayhoff et al. (1978) Atlas of Protein Sequence and Structure (Natl. Biomed. Res. Found., Washington, D.C.), herein incorporated by reference. Conservative substitutions, such as exchanging one amino acid with another having similar properties, may be optimal.

Methods of alignment of sequences for comparison are well known in the art. Thus, the determination of percent sequence identity between any two sequences can be accomplished using a mathematical algorithm. Non-limiting examples of such mathematical algorithms are the algorithm of Myers and Miller, (1988) CABIOS 4:11-17; the algorithm of Smith, et al., (1981) Adv. Appl. Math. 2:482; the algorithm of Needleman and Wunsch, (1970) J. Mol. Biol. 48:443-453; the algorithm of Pearson and Lipman, (1988) Proc. Natl. Acad. Sci. 85:2444-2448; the algorithm of Karlin and Altschul, (1990) Proc. Natl. Acad. Sci. USA 872:264, modified as in Karlin and Altschul, (1993) Proc. Natl. Acad. Sci. USA 90:5873-5877, herein incorporated by reference in their entirety. Computer implementations of these mathematical algorithms are well known in the art and can be utilized for comparison of sequences to determine sequence identity.

As used herein, “sequence identity” or “identity” in the context of two nucleic acid or polypeptide sequences refers to the residues in the two sequences that are the same when aligned for maximum correspondence over a specified comparison window. When percentage of sequence identity is used in reference to proteins it is recognized that residue positions which are not identical often differ by conservative amino acid substitutions, where amino acid residues are substituted for other amino acid residues with similar chemical properties (e.g., charge or hydrophobicity) and therefore do not change the functional properties of the molecule. When sequences differ in conservative substitutions, the percent sequence identity may be adjusted upwards to correct for the conservative nature of the substitution. Sequences that differ by such conservative substitutions are said to have “sequence similarity” or “similarity”. Means for making this adjustment are well known to those of skill in the art. Typically, this involves scoring a conservative substitution as a partial rather than a full mismatch, thereby increasing the percentage sequence identity. Thus, for example, where an identical amino acid is given a score of one and a non-conservative substitution is given a score of zero, a conservative substitution is given a score between zero and one. The scoring of conservative substitutions is calculated, e.g., as implemented in the program PC/GENE (Intelligenetics, Mountain View, Calif.).

As used herein, “percentage of sequence identity” means the value determined by comparing two optimally aligned sequences over a comparison window, wherein the portion of the polynucleotide sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison, and multiplying the result by 100 to yield the percentage of sequence identity.

The term “substantial identity” of polynucleotide sequences means that a polynucleotide comprises a sequence that has at least 70% sequence identity, optimally at least 80%, more optimally at least 90% and most optimally at least 95%, compared to a reference sequence using an alignment program using standard parameters. One of skill in the art will recognize that these values can be appropriately adjusted to determine corresponding identity of proteins encoded by two nucleotide sequences by considering codon degeneracy, amino acid similarity, reading frame positioning and the like. Substantial identity of amino acid sequences for these purposes normally means sequence identity of at least 60%, 70%, 80%, 90% and at least 95%.

Genes included in expression vectors must be driven by a nucleotide sequence comprising a regulatory element, for example, a promoter. Several types of promoters are now well known in the transformation arts, as are other regulatory elements that can be used alone or in combination with promoters.

For example, if the transgenic polynucleotide of interest is to be used to separate transgenic seed from non-transgenic seed, a non-lethal marker such as a visually scoreable color marker that expresses at detectable, preferably high levels, in the seed may be desirable.

As used herein, the term “expression cassette” means a distinct component of vector DNA consisting of coding and non-coding sequences including 5′ and 3′ regulatory sequences that control expression in a transformed/transfected cell.

As used herein, the term “coding sequence” means the portion of DNA sequence bounded by a start and a stop codon that encodes the amino acids of a protein.

As used herein, the term “non-coding sequence” means the portions of a DNA sequence that are transcribed to produce a messenger RNA, but that do not encode the amino acids of a protein, such as 5′ untranslated regions, introns and 3′ untranslated regions. Non-coding sequence can also refer to RNA molecules such as micro-RNAs, interfering RNA or RNA hairpins, that when expressed can down-regulate expression of an endogenous gene or another transgene.

As used herein, the term “regulatory sequence” or “regulatory element” also refers to a sequence of DNA, usually, but not always, upstream (5′) to the coding sequence of a structural gene, which includes sequences which control the expression of the coding region by providing the recognition for RNA polymerase and/or other factors required for transcription to start at a particular site. An example of a regulatory element that provides for the recognition for RNA polymerase or other transcriptional factors to ensure initiation at a particular site is a promoter element. A promoter element comprises a core promoter element, responsible for the initiation of transcription, as well as other regulatory elements that modify gene expression. It is to be understood that nucleotide sequences, located within introns or 3′ of the coding region sequence may also contribute to the regulation of expression of a coding region of interest. Examples of suitable introns include, but are not limited to, the maize IVS6 intron, the maize actin intron, or the maize shrunken 1 sucrose synthase intron. A regulatory element may also include those elements located downstream (3′) to the site of transcription initiation, or within transcribed regions, or both. In the context of the methods of the disclosure, a post-transcriptional regulatory element may include elements that are active following transcription initiation, for example translational and transcriptional enhancers, translational and transcriptional repressors and mRNA stability determinants.

The term “operably linked” refers to a functional linkage between a promoter or other regulatory element and an associated transcribable DNA sequence or coding sequence of a gene (or transgene), such that the promoter, etc., operates or functions to initiate, assist, affect, cause, and/or promote the transcription and expression of the associated transcribable DNA sequence or coding sequence, at least in certain cell(s), tissue(s), developmental stage(s), and/or condition(s).

The term “heterologous” in reference to a promoter or other regulatory sequence in relation to an associated polynucleotide sequence (e.g., a transcribable DNA sequence or coding sequence or gene) is a promoter or regulatory sequence that is not operably linked to such associated polynucleotide sequence in nature—e.g., the promoter or regulatory sequence has a different origin relative to the associated polynucleotide sequence and/or the promoter or regulatory sequence is not naturally occurring in a plant species to be transformed with the promoter or regulatory sequence.

A “heterologous nucleotide sequence”, “heterologous polynucleotide of interest”, or “heterologous polynucleotide” as used throughout the disclosure, is a sequence that is not naturally occurring with or operably linked to a promoter. While this nucleotide sequence is heterologous to the promoter sequence, it may be homologous or native or heterologous or foreign to the plant host. Likewise, the promoter sequence may be homologous or native or heterologous or foreign to the plant host and/or the polynucleotide of interest.

The term “recombinant” in reference to a polynucleotide (DNA or RNA) molecule, protein, construct, vector, etc., refers to a polynucleotide or protein molecule or sequence that is man-made and not normally found in nature, and/or is present in a context in which it is not normally found in nature, including a polynucleotide (DNA or RNA) molecule, protein, construct, etc., comprising a combination of two or more polynucleotide or protein sequences that would not naturally occur together in the same manner without human intervention, such as a polynucleotide molecule, protein, construct, etc., comprising at least two polynucleotide or protein sequences that are operably linked but heterologous with respect to each other. For example, the term “recombinant” can refer to any combination of two or more DNA or protein sequences in the same molecule (e.g., a plasmid, construct, vector, chromosome, protein, etc.) where such a combination is man-made and not normally found in nature. As used in this definition, the phrase “not normally found in nature” means not found in nature without human introduction. A recombinant polynucleotide or protein molecule, construct, etc., may comprise polynucleotide or protein sequence(s) that is/are (i) separated from other polynucleotide or protein sequence(s) that exist in proximity to each other in nature, and/or (ii) adjacent to (or contiguous with) other polynucleotide or protein sequence(s) that are not naturally in proximity with each other. Such a recombinant polynucleotide molecule, protein, construct, etc., may also refer to a polynucleotide or protein molecule or sequence that has been genetically engineered and/or constructed outside of a cell. For example, a recombinant DNA molecule may comprise any engineered or man-made plasmid, vector, etc., and may include a linear or circular DNA molecule. Such plasmids, vectors, etc., may contain various maintenance elements including a prokaryotic origin of replication and selectable marker, as well as one or more transgenes or expression cassettes perhaps in addition to a plant selectable marker gene, etc.

Transformation protocols as well as protocols for introducing nucleotide sequences into plants may vary depending on the type of plant or plant cell, i.e., monocot or dicot, targeted for transformation. Suitable methods of introducing nucleotide sequences into plant cells and subsequent insertion into the plant genome include microinjection, electroporation direct gene transfer, and ballistic particle acceleration.

In an aspect, the present disclosure comprises compositions, methods of making such compositions, as well as, methods of using such compositions for producing a modified plant. The term “plant” refers to whole plants, plant organs (e.g., leaves, stems, roots, etc.), plant tissues, plant cells, plant parts, seeds, propagules, embryos and progeny of the same. Plant cells can be differentiated or undifferentiated (e.g. callus, undifferentiated callus, immature and mature embryos, immature zygotic embryo, immature cotyledon, embryonic axis, suspension culture cells, protoplasts, leaf, leaf cells, root cells, phloem cells and pollen). Plant cells include, without limitation, cells from seeds, suspension cultures, explants, immature embryos, embryos, zygotic embryos, somatic embryos, embryogenic callus, meristem, somatic meristems, organogenic callus, protoplasts, embryos derived from mature ear-derived seed, leaf bases, leaves from mature plants, leaf tips, immature inflorescences, tassel, immature ear, silks, cotyledons, immature cotyledons, embryonic axes, meristematic regions, callus tissue, cells from leaves, cells from stems, cells from roots, cells from shoots, gametophytes, sporophytes, pollen and microspores. Plant parts include differentiated and undifferentiated tissues including, but not limited to, roots, stems, shoots, leaves, pollen, seeds, tumor tissue and various forms of cells in culture (e. g., single cells, protoplasts, embryos, and callus tissue). The plant tissue may be in a plant or in a plant organ, tissue, or cell culture. Grain is intended to mean the mature seed produced by commercial growers for purposes other than growing or reproducing the species. Progeny, variants and mutants of the regenerated plants are also included within the scope of the disclosure, provided these progeny, variants and mutants comprise the introduced polynucleotides.

Agrobacterium strains are useful for the genetic engineering of plants, e.g. to produce a transformed or transgenic plant, to express a phenotype of interest. As used herein, the terms “transformed plant” and “transgenic plant” refer to a plant that comprises within its genome a heterologous polynucleotide. Generally, the heterologous polynucleotide is stably integrated within the genome of a transgenic or transformed plant such that the polynucleotide is passed on to successive generations. The heterologous polynucleotide may be integrated into the genome alone or as part of a recombinant DNA construct. It is to be understood that as used herein the term “transgenic” includes any cell, cell line, callus, tissue, plant part or plant the genotype of which has been altered by the presence of a heterologous nucleic acid including those transgenics initially so altered as well as those created by sexual crosses or asexual propagation from the initial transgenic.

Cells that have been transformed may be grown into plants in accordance with conventional ways. See, for example, McCormick, et al., (1986) Plant Cell Reports 5:81-84, herein incorporated by reference in its entirety. These plants may then be grown, and either pollinated with the same transformed strain or different strains, and the resulting progeny having expression of the desired phenotypic characteristic identified. Two or more generations may be grown to ensure that expression of the desired phenotypic characteristic is stably maintained and inherited and then seeds harvested to ensure expression of the desired phenotypic characteristic has been achieved. In this manner, the present disclosure provides transformed seed (also referred to as “transgenic seed”) having an expression cassette useful in the methods of the disclosure stably incorporated into its genome.

Methods are known in the art for the targeted insertion of a polynucleotide at a specific location in the plant genome. The insertion of the polynucleotide at a desired genomic location is achieved using a site-specific recombination system. See, for example, U.S. Pat. Nos. 9,222,098 B2, 7,223,601 B2, 7,179,599 B2, and 6,911,575 B1, all of which are herein incorporated by reference in their entirety.

As used herein, a “targeted genome editing technique” refers to any method, protocol, or technique that allows the precise and/or targeted editing of a specific location in a genome of a plant (i.e., the editing is largely or completely non-random) using a site-specific nuclease, such as a meganuclease, a zinc-finger nuclease (ZFN), an RNA-guided endonuclease (e.g., the CRISPR/Cas9 system), a TALE-endonuclease (TALEN), a recombinase, or a transposase. See, e.g., Khandagale, K. et al. (2016) “Genome editing for targeted improvement in plants,” Plant Biotechnol Rep 10: 327-343; and Gaj, T. et al. (2013) “ZFN, TALEN and CRISPR/Cas-based methods for genome engineering,” Trends Biotechnol. 31(7): 397-405. As used herein, “editing” or “genome editing” refers to generating a targeted mutation, deletion, inversion or substitution of at least 1, at least 2, at least 3, at least 4, at least 5, at least 6, at least 7, at least 8, at least 9, at least 10, at least 15, at least 20, at least 25, at least 30, at least 35, at least 40, at least 45, at least 50, at least 75, at least 100, at least 250, at least 500, at least 1000, at least 2500, at least 5000, at least 10,000, or at least 25,000 nucleotides of an endogenous plant genome nucleic acid sequence. As used herein, “editing” or “genome editing” also encompasses the targeted insertion or site-directed integration of at least 1, at least 2, at least 3, at least 4, at least 5, at least 6, at least 7, at least 8, at least 9, at least 10, at least 15, at least 20, at least 25, at least 30, at least 35, at least 40, at least 45, at least 50, at least 75, at least 100, at least 250, at least 500, at least 750, at least 1000, at least 1500, at least 2000, at least 2500, at least 3000, at least 4000, at least 5000, at least 10,000, or at least 25,000 nucleotides into the endogenous genome of a plant. An “edit” or “genomic edit” in the singular refers to one such targeted mutation, deletion, inversion, substitution or insertion, whereas “edits” or “genomic edits” refers to two or more targeted mutation(s), deletion(s), inversion(s), substitution(s) and/or insertion(s), with each “edit” being introduced via a targeted genome editing technique.

In an aspect, Agrobacterium transformation can be used to introduce into plants polynucleotides that are useful to target a specific site for modification in the genome of a plant or plant cell. Site specific modifications that can be introduced using Agrobacterium transformation, for example, include those produced using any method for introducing site specific modification, including, but not limited to, through the use of gene repair oligonucleotides (e.g. US Publication 2013/0019349), or through the use of double-stranded break technologies such as TALENs, meganucleases, zinc finger nucleases, CRISPR-Cas, and the like. For example, targeted genome editing methods, using Agrobacterium transformation, can be used to introduce a CRISPR-Cas system into a plant cell or plant, for the purpose of genome modification of a target sequence in the genome of a plant or plant cell, for selecting plants, for deleting a base or a sequence, for gene editing, and for inserting a polynucleotide of interest into the genome of a plant or plant cell. Thus, targeted genome editing methods, using Agrobacterium transformation, can be used together with a CRISPR-Cas system to provide for an effective system for modifying or altering target sites and nucleotides of interest within the genome of a plant, plant cell or seed. The Cas endonuclease gene is a plant optimized Cas9 endonuclease, wherein the plant optimized Cas9 endonuclease is capable of binding to and creating a double strand break in a genomic target sequence of the plant genome.

Also provided herein is a modified wheat plant, seed, or plant cell comprising one of the plant-derived polynucleotides encoding screenable markers operably linked to a promoter that expresses in seed. In some aspects, the plant-derived polynucleotides encoding screenable markers operably linked to a promoter that expresses in seed is also operably linked to a male-fertility restoration polynucleotide driven by a male-tissue specific promoter.

In some examples, the plant-derived polynucleotide encoding a screenable marker is edited to be driven by a promoter that expresses in seed, including swapping promoters with the native promoter, or has a polynucleotide sequence inserted so that the screenable marker is expressed in seed specifically or preferentially. In some aspects, this may include editing or inserting a regulatory element, such as an enhancer or intron sequence, to enhance expression of polynucleotide encoding the screenable marker in seed, e.g. either to boost a seed-specific or seed-preferred promoter or directly cause express in seed by itself. In one example, a native promoter of a wheat P1 gene on 4A chromosome may be swapped with the promoter of a seed-specific wheat gene. In another example, an appropriate expression enhancing element may be inserted into the promoter of Ta-P1-4A to render seed specificity to the native P1 gene.

In some aspects, the polynucleotide encoding the screenable marker includes a nucleotide sequence of SEQ ID NO:1, 3, 5, 7, or 9, its variants, or fragments thereof; a nucleotide sequence that is at least 80%, at least 85%, at least 90%, at least 95%, at least 96%, at least 97%, at least 98%, or at least 99% sequence identical to the nucleotide sequence of SEQ ID NO: 1, 3, 5, 7, or 9, its variants, or fragments thereof; a nucleotide that encodes a polypeptide with an amino acid sequence of SEQ ID NO: 2, 4, 6, 8, or 10; or a nucleotide that encodes a polypeptide that is at least 85%, 90%, 91%, 92%, 93%, 94%, 95%, 96%, 97%, 98%, or 99% identical to the amino acid sequence of SEQ ID NO: 2, 4, 6, 8, or 10. In some aspects, the percent identity is determined with respect to the full length nucleotide sequence of SEQ ID NO: 1, 3, 5, 7, or 9 or the full length amino acid sequence of SEQ ID NO: 2, 4, 6, 8, or 10. In some aspects, the nucleic acid fragments encode a screenable marker that comprises at least 200, 300, or 400 contiguous amino acids of the polypeptides of SEQ ID NO: 2, 4, 6, 8, or 10. In some aspects, the polynucleotide encoding the screenable marker operably linked to a heterologous promoter that expresses in seed is included in a recombinant DNA construct.

The modified wheat seed disclosed herein is characterized by having a change in seed color, opacity, intensity, or other seed property relative to an unmodified isogenic wheat seed lacking expression of the plant-derived polynucleotide encoding a screenable marker in seed as disclosed elsewhere herein.

In one aspect, the mixture of seeds may be separated, if desired. Seeds that contain the polynucleotides of interest may be identified using any suitable methods or techniques. Examples include, but are not limited to, molecular marker analysis, phenotype analysis, PCR, progeny tests, molecular markers, or ELISA could be used to trace the transgenic polynucleotides of interest. For example, in one aspect, the recombinant DNA construct may contain in addition to the one or more male-fertility restoration polynucleotides and the polynucleotide encoding the screenable marker that expresses in seed, a polynucleotide that when expressed inhibit pollen function or formation to prevent transmission of the DNA construct in pollen, for example, alpha amylase. Such construct may be used to create a maintainer.

Seeds that contain the polynucleotide of interest, e.g. the polynucleotide encoding the screenable marker, and those seeds that do not, may be identified and separated by color where seeds expressing the color marker (for example, with respect to the P1 gene, a dark brown color) indicate that those seeds contain the polynucleotide of interest. In one aspect, the seeds are identified for the color marker and separated using a sorting machine. The sorting may be performed by any suitable method. For example, the seeds may be separated visually. This may be accomplished using a seed sorter or using a spectrophotometer that measures a particular wavelength to separate fluorescent color markers such as green, yellow, red fluorescent protein. Populations of seeds may be sorted using optical sensing technology including multi or hyper spectral imaging, UV, visible or NIR spectroscopy systems, and/or optical scanning.

A modified wheat plant can be generated from the modified wheat plant cell or seed disclosed herein that comprises the plant-derived polynucleotide encoding a screenable marker as described elsewhere herein.

The color genes of this invention can be used as a screenable marker gene in any situation in which it is worthwhile to detect the presence of a foreign DNA (i.e. a transgene) in seeds of a transformed plant in order to isolate seeds which possess the foreign DNA. In this regard virtually any foreign DNA can be linked to the color gene. Examples of such foreign DNAs are genes coding for insecticidal (e.g. from Bacillus thuringiensis), fungicidal or nematocidal proteins. Similarly, the screenable marker gene can be linked to a foreign DNA which is a male-fertility restorer gene. In appropriate conditions the use of the color genes allows the easy separation of harvested seeds that will grow into male-sterile plants, and harvested seeds that will grow into male-fertile plants. In this regard the seeds are preferably harvested from male-sterile plants that are homozygous at a male-sterility locus.

All publications and patent applications mentioned in the specification are indicative of the level of those skilled in the art to which this disclosure pertains, and all such publications and patent applications are herein incorporated by reference to the same extent as if each individual publication or patent application was specifically and individually indicated to be incorporated by reference.

### EXAMPLES

In the following Examples, unless otherwise stated, parts and percentages are by weight and degrees are Celsius. It should be understood that these Examples, while indicating embodiments of the disclosure, are given by way of illustration only. From the above discussion and these Examples, one skilled in the art can make various changes and modifications of the disclosure to adapt it to various usages and conditions. Such modifications are also intended to fall within the scope of the appended claims.

Traditionally, proteins imparting fluorescence to tissues such DSRED, CFP, YFP and GFP have been used as markers to follow transgenes. Anthocyanins and flavonoids are pigments produced by plants for coloration of various tissues and organs. The utilization of such a plant-based marker can facilitate the assembly of a DsRed-free system for production of hybrid wheat seed and can be used for trait discovery purposes where seed markers are required.

### Example 1: A Mutant Allelic Variant of the Maize P1 Gene Expressed with Seed-Specific Promoters Imparted Color to Wheat Seeds

The maize Pericarp color1 (P1) gene regulates the flavonoid biosynthesis pathway and imparts color to the seed pericarp and other vegetative tissues of the plant (Cocciolone et al., 2001, Plant J., 27(5):467-78). The P1 protein is 376 amino acid and consists of a MYB DNA binding domain and P-protein C-terminus domain (Grotewald et al., 1994, Cell, 76, 543-553). Several allelic variants of P1 have been produced through transposon-based mutagenesis (Zhang and Peterson, 2005, The Plant Cell, 17:903-914). An allelic variant, P1-trunc, (SEQ ID NO: 3) derived through mutagenesis retains both the MYB and P-protein domains but the sequence downstream of amino acid (aa) position 250 is changed and is 41 aa shorter due to introduction of a stop codon resulting in a mutated-truncated P1 protein. The maize P1 full length gene (SEQ ID NO: 1) and P1-trunc were tested in wheat through expression with various seed-specific promoters and enhancer elements for their ability to impart color to wheat seeds. Table 2 lists the different gene, promoter, and enhancer combinations.

TABLE 2

| Vector | Description | T0 Phenotype |
|---|---|---|
| V0001 | LTP2 PRO:ZM-P1-trunc | Seeds segregating for dark brown seed color |
| V0002 | 35SENH-LTP2 PRO:ZM-P1-trunc | Seeds segregating for dark brown seed color. |
| | | Enhanced color expression in the aleurone. |
| V0003 | LTP2 PRO:ZM-SH1-INT:ZM-P1- | Seeds segregating for dark brown seed color. |
| | trunc | Enhanced color expression in the aleurone. |
| V0004 | 35S ENH:CZ19B1 PRO:ZM-P1-trunc | Seeds segregating for dark brown seed color. |
| | | Enhanced color expression in the aleurone. |
| V0005 | 35S ENELGZ-W64A PRO:P1-trunc | Seeds segregating for dark brown seed color. |
| | | Enhanced color expression in the aleurone. |
| V0006 | 35S ENH:LTP2 PRO:TA-P1-4A | Seeds segregating for dark brown color |
| V0007 | 35S ENH:LTP2 PRO:TA-P1-1D | Seeds did not exhibit or segregate for color |
| V0008 | 35S ENH:LTP2 PRO:P1-trunc - | Seeds segregating 50:50 for color:non-color |
| | PG47:Alpha Amylase - Ms1 | |
| V0009 | LTP2 PRO:OS-KALA4 | Seeds segregated for dark-shaded seeds |

In vector V0001, the truncated version of P1 was transcriptionally fused to the aleurone-specific LTP2 promoter and transformed into wheat genotypes Fielder (soft white wheat) and SBC0456D (hard red wheat), using standard transformation methods. See, for example, He, et al., (2010) J. Exp. Botany 61(6):1567-1581; Wu, et al., (2008) Transgenic Res. 17:425-436; Nehra, et al., (1994) Plant J. 5(2):285-297; Rasco-Gaunt, et al., (2001) J. Exp. Botany 52(357):865-874; Razzaq, et al., (2011) African J. Biotech. 10(5):740-750; Tamás-Nyitrai, et al., (2012) Plant Cell Culture Protocols, Methods in Molecular Biology 877:357-384; and U.S. patent publication 2014/0173781.

T0 plants were regenerated and genotyped for vector T-DNA copy number and plants with intact single T-DNA insertions were grown to maturity, seeds were harvested and analyzed. T0 plants generated from independent T-DNA insertion events showed consistent dark brown seed color phenotype. Seed color segregated in the manner consistent with seed-specific expression. The seed color was stably inherited across generations and was observed similarly segregating in seeds harvested from T1 plants. The dark brown P1-trunc expressing seed also exhibited higher fluorescence compared to non-transformed when observed with GFP optimized filters. Importantly, P1-trunc expression and the accumulated pigment did not have any effect of seed development and seed germination. No seed phenotypes were observed with the full-length version of the P1 gene.

In vector V0002, an enhancer element from the CAMV 35S promoter (CAMV 35S ENH) was fused to the LTP2 promoter to drive the expression of P1-trunc. The T0 and T1 plants generated from this construct showed boosted color expression in the seed aleurone. Similarly, in vector V0003, the LTP2 promoter was fused to ZM-SH1-INT, the intron of maize shrunken 1 sucrose synthase gene (dpzm09g004800.1.2) to drive the expression of P1-trunc gene. ZM-SH1-INT is able to enhance tissue-specific gene expression when fused to promoters. See, for example, PCT patent publication WO2018183878, published Oct. 4, 2018, incorporated herein by reference in its entirety. The T0 plants generated from vector V0003 showed enhanced color expression in the seed aleurone. These results suggested that various enhancer elements can be utilized to enhance expression of P1-trunc gene.

Two additional seed-specific promoters were also tested for expression of P1-trunc gene. In vector V0004, the promoter of the maize 19KD B1 Zein gene (CZ19B1) was fused to the CAMV 35S ENH to drive the expression of the P1-trunc gene. Similarly, in vector V0005, the promoter of maize 27 KD Gamma zein gene (GZ-W64A) was fused to the CAMV 35S ENH to drive the expression of P1-trunc gene. The T0 plants generated with vector V0004 and V0005 also exhibited color expression in the seed aleurone suggesting that a variety of seed-specific promoters can be used to drive the P1-trunc gene expression to achieve seed coloration.

### Example 2: A Native Wheat P1 Gene Expressed with Seed-Specific Promoters Imparts Color to Wheat Seeds

Wheat homologs of the maize P1 gene were also tested for seed color expression in wheat. The proteins encoded by P1 genes on chromosome 4 homolog group (4AS, 4BL and 4DL) were the most similar to the maize P1 and P1-trunc proteins with 46-51% amino acid identity. The three group 4 wheat P1 homologs were 98% identical to each other in amino acid composition. When the first 250 amino acid sequence, which contains the MYB and the P-domains, was compared, the identity between the maize P1 and wheat homologs increased to 64-65%. The second group of proteins encoded by P1 genes on chromosome 1 (1AL, 1BS and 1DS) were 37-46% identical to the maize P1 and P1-trunc protein.

Ta-P1-4A (SEQ ID NO:5) from the homolog group chromosome 4 and Ta-P1-1D (SEQ ID NO:7) from homolog group chromosome 1 were selected for further analysis through expression in seeds. CAMV 35S ENH and LTP2 were fused to drive the expression of Ta-P1-4A and Ta-P1-1D to make vectors V0006 and V0007, respectively. These vectors were transformed into wheat genotypes Fielder (soft white wheat) and SBC0456D (hard red wheat). T0 plants were regenerated and genotyped for vector T-DNA and plants with intact single T-DNA insertions were grown to maturity, seeds were harvested and analyzed. It was observed that plants transformed with V0006 produced seeds that segregated for seed color similar to the plants with vectors V0001 and V0002. Seeds harvested from plants transformed with vectors V0007 did not exhibit or segregate for color. These observations strongly suggest that the Ta-P1-4A has the same properties as maize P1-trunc and can be utilized similarly to maize P1-trunc to impart seed color to wheat seeds.

### Example 3: A P1 Gene Expressed with Seed-Specific Promoters can be Used as a Marker to Maintain Male Sterile Inbreds for Hybrid Seed Production

P1 gene versions, such as the P1-trunc or Ta-P1-4A can be used to constitute a hybrid seed production system to identify seed that will produce male sterile plants very similar to those using DSRED described in Wu et al. 2016, Plant Biotechnology Journal, 14: 1046-54, but with the advantage of being a plant protein from a grass species (P1-trunc) or a wheat-specific protein (Ta-P1-4A).

Utilizing the P1-trunc, alpha amylase and Ms1 polynucleotides, vectors were constructed that were utilized in combination with ms1d mutations (Tucker et al., 2017, Nature Communications, 8: 869) for a wheat hybrid seed production system. In vector V0008, P1-trunc was transcriptionally fused to CAMV 35S enhancer and LTP2 promoter. Alpha amylase polynucleotide was transcriptionally fused to the maize PG47 promoter. In addition, this vector also included Ms1 genomic fragment which comprised the native promoter and terminator fragment in addition to the Ms1 gene. These vectors were transformed into wheat genotype SBC0456D (hard red wheat). T0 plants were regenerated and genotyped for vector T-DNA and plants with intact single T-DNA insertions were grown to maturity, seeds were harvested and analyzed. Since the alpha amylase degrades starch in pollen and renders it non-functional, the vector T-DNA was expected to be transmitted only through the female gametes. Due to this transmission pattern the seeds were expected to segregate 50:50 for color:non-color. It was observed that seeds from 1-copy T0 plants segregated 50:50 for color:non-color. T1 plants were grown, self-pollinated and seeds were analyzed. These seeds also segregated 50:50 for color:non-color suggesting stable inheritance of T-DNA and P1-trunc induced seed color phenotype.

T1 plants containing TDNA cassette from vector V0008 were crossed as females to plants carrying the ms1d mutation in a heterozygous state. F1 plants were grown and self-pollinated to obtain F2 seeds that segregated for color:non-color seeds and ms1d mutation. A set of plants were grown from colored and non-colored seeds. The F2 plants were genotyped for the ms1d mutation and plants homozygous for ms1d were identified from both the colored and non-colored seeds. The ms1d homozygous plants generated from colored seeds were fertile whereas the plants from non-colored seeds were male sterile (Table 3).

TABLE 3

| Plant # | Seed Color | V0008 TDNA | Male fertility | Utility |
|---|---|---|---|---|
| 1 | Yes | 1-copy | Fertile | Maintainer for next generation |
| 2 | Yes | 1-copy | Fertile | Maintainer for next generation |
| 3 | Yes | 1-copy | Fertile | Maintainer for next generation |
| 4 | No | No TDNA | Sterile | Female for hybrid seed production |
| 5 | No | No TDNA | Sterile | Female for hybrid seed production |
| 6 | No | No TDNA | Sterile | Female for hybrid seed production |

These observations showed that it is possible to utilize P1 gene as a color marker for assembling a maintainer inbred for hybrid seed production system. The self-pollinated seed from the maintainer will segregate for seed color and it is possible to identify seed that will produce male fertile or male sterile plants. The male sterile plants generated from non-colored seeds can be used as female parent in a hybrid seed production. This data clearly demonstrated that the P1 can be used as a seed marker in a maintainer line for maintenance of male sterility.

The seeds expressing P1-trunc or Ta-P1-4A can be mechanically sorted using a variety of seed sorters. To test this hypothesis, a RBG analytic color sorter (VMEK, Midlothian, Va.) was used. This seed sorter was able to efficiently sort P1-trunc expressing dark brown seeds from red or white wheat seeds without P1-trunc. Thus, the currently available color sorting technology can be used to sort seeds expressing P1-trunc color from non-color expressing seeds. The P1-trunc expressing dark brown seed also exhibited higher fluorescence compared to non-transformed (Example 1). The RBG sorting technology is combined with fluorescence sorting technology to further increase efficiency of seed sorting.

### Example 4: Utilizing the Native Wheat P1 Gene on Chromosome 4L to Create a Maintainer Chromosome Through Gene Editing

The Ta-P1-4A gene resides on the same chromosome arm (4L) as the wheat fertility genes Ms45, Ms26 and Ms9, and thus is linked to these genes. The most tightly linked gene to Ta-P1-4A is TaMs9 with a distance of 7.7 Mb between them. It is possible to exploit this genetic linkage of the Ta-P1-4A gene to male fertility genes to reconstruct a male sterility maintainer system in wheat such as that described in Example 3.

The expression of the native wheat Ta-P1-4A gene would need to be modulated, possibly through CRISPR-mediated genome editing, to create a maintainer chromosome. The native promoter of Ta-P1-4A gene can be swapped with the promoter of an endosperm-specific wheat gene, or alternatively an appropriate expression enhancing element can be inserted into the promoter of Ta-P1-4A that can render seed specificity. This can be achieved for example, through insertion of an endosperm-specific promoter such as Zea mays LTP2 promoter before the native wheat P1 gene, or the replacement of native wheat P1 promoter with Zm-LTP2 promoter. This is also known as a promoter swap. This manipulation will generate a maintainer chromosome which will function similarly as the TDNA cassette described in Example 3 when combined with a suitable mutation in any of the linked male fertility genes. The seeds from such a maintainer will segregate 3:1 for colored and non-colored seeds. The non-colored seeds will generate male sterile plants similar to those described in Example 3.

### Example 5: Enhancer Element Insertions or Deletions Using the guideRNA/Cas Endonuclease System

The guide RNA/Cas endonuclease system described herein can be used to allow for the insertion or deletion of a promoter element from either a transgenic (pre-existing, artificial) or endogenous gene. Promoter elements, such as enhancer elements, are often introduced in promoters driving gene expression cassettes in multiple copies (3×=3 copies of enhancer element) for trait gene testing or to produce transgenic plants expressing specific trait. Enhancer elements can be, but are not limited to, SV40 enhancer region and the 35S enhancer element. In some plants (events), the enhancer elements can cause an unwanted phenotype, a yield drag, or a change in expression pattern of the trait of interest that is not desired. It may be desired to insert or remove extra copies of the enhancer element while keeping the trait gene cassettes intact at their integrated genomic location. The guide RNA/Cas endonuclease system described herein can be used to insert a desired enhancing element or to remove an unwanted enhancing element from the plant genome. A guide RNA can be designed to contain a variable targeting region targeting a target site sequence of 12-30 bps adjacent to a NGG (PAM) in the enhancer. The Cas endonuclease can make cleavage to insert or remove one or multiple enhancers. The guideRNA/Cas endonuclease system can introduced by either Agrobacterium or particle gun bombardment. Alternatively, two different guide RNAs (targeting two different genomic target sites) can be used to insert or remove one or more enhancer elements into or from the genome of an organism, in a manner similar to the insertion or removal of a (transgenic or endogenous) promoter described herein.

### Example 6: Improving Linkage Between the Wheat P1 Gene and the Wheat Fertility Genes Using the guideRNA/Cas Endonuclease System

One of the most important characteristics of the maintainer chromosome mentioned in Example 4 is the lack of recombination between the male fertility gene and the color marker gene (Ta-P1-4A). While the most tightly linked gene to Ta-P1-4A is TaMs9 with a distance of 7.7 Mb between them, Ms45 and Ms26 genes are more distantly placed compared to Ta-P1-4A and therefore are less tightly linked. To effectively utilize Ms45 and Ms26 genes to create maintainer chromosome, or to further tighten the linkage between Ms9 and Ta-P1-4A, the distance between the fertility genes and Ta-P1-4A can be reduced to create a tighter linkage. This can be achieved both through native physical mutagenesis techniques such as gamma radiation or using the genome editing techniques (e.g., CRISPR-Cas). Utilizing CRISPR-mediated genome editing, a large deletion between any of the male fertility genes and Ta-P1-4A can be created as shown by Li et al., in Plant Genome Editing with CRISPR Systems, 2019, pp. 47-61, Humana Press, NY. Creating such a deletion would bring the two genes physically closer creating a tight linkage.

### Example 7: Targeting Additional Chromosomes with Maize P1 Gene Variants Using the guideRNA/Cas Endonuclease System

The utilization of Ta-P1-4A as a marker to maintain male sterile inbreds, as outlined in Example 3, can be further expanded to specifically place the Ms1-P1 marker gene TDNA on a chromosome from an alien species, including but not limited to the 4E, 4EL, or 4H chromosome from Thinopyrum, Aegilops, Secale, Haynaldia, Elyymus, or Hordeum, that has been introduced into wheat through traditional breeding. Such modification would not alter the genomic composition of wheat chromosomes but will provide the benefits of the maintainer system as outlined in Example 3. The addition of an extra chromosome in wheat to create a monosomic addition line. Monosomics segregate in a non-Mendelian pattern. However, the monosomics do however produce disomoics at some frequency, which can fix the maintainer genotype. The added advantage of this system would be the elimination of the production of disomics.

### Example 8: Seed-Specific Expression of the Rice Kala4 Gene can Impart Color to Wheat Seeds

Kala4 gene is a basic Helix-Loop-Helix (bHLH) transcription factor that regulates anthocyanin biosynthesis pathway in rice. Ectopic seed-specific expression of Kala4 gene produces black seed phenotype in rice (Oikawa et al., 2015). We tested if Kala4 can induce a seed color phenotype in wheat. In vector V0009 LTP2 promoter was transcriptionally fused to the rice Kala4 genomic sequence, excluding the Kala4 promoter, and transformed into wheat genotypes Fielder (soft white wheat) and SBC0456D (hard red wheat). T0 plants were regenerated and genotyped for vector TDNA and plants with intact single T-DNA insertions were grown to maturity. Seed-specific color was observed in the developing wheat seeds. At maturity the seeds segregated for dark shaded seeds and light seeds which did not have the T-DNA insertion. These observations showed that the rice Kala4 gene can be utilized as a potential plant screenable marker for wheat seeds.

We claim:

1. A polynucleotide encoding a screenable marker for seed selection, wherein the polynucleotide is selected from the group consisting of:
a) a nucleotide sequence of SEQ ID NO:1, 3, 5, 7, or 9;
b) a nucleotide sequence that is at least 85% identical to the nucleotide sequence of SEQ ID NO: 1, 3, 5, 7, or 9;
c) a nucleotide fragment of the nucleotide sequence of part a;
d) a nucleotide fragment of the nucleotide sequence of part b;
e) a nucleotide that encodes a polypeptide with an amino acid sequence of SEQ ID NO: 2, 4, 6, 8, or 10;
f) a nucleotide that encodes a polypeptide that is at least 85% identical to the amino acid sequence of SEQ ID NO: 2, 4, 6, 8, or 10;
wherein the polynucleotide is operably linked to a promoter that expresses in seed.

2. A recombinant DNA construct comprising the polynucleotide of claim 1.

3. A seed comprising the polynucleotide of claim 1.

4. A method of restoring male fertility in a male-sterile plant, the method comprising:
a) introducing into a male-sterile plant, wherein the male-sterile plant comprises one or more homozygous mutations in an endogenous male-fertility polynucleotide that confers male sterility to the plant, one or more male-fertility restoration polynucleotides operably linked to a polynucleotide encoding a screenable marker for seed selection, wherein the polynucleotide is selected from the group consisting of:
i. a nucleotide sequence of SEQ ID NO:1, 3, 5, 7, or 9;
ii. a nucleotide sequence that is at least 85% identical to the nucleotide sequence of SEQ ID NO: 1, 3, 5, 7, or 9;
iii. a nucleotide fragment of the nucleotide sequence of part a;
iv. a nucleotide fragment of the nucleotide sequence of part b;
v. a nucleotide that encodes a polypeptide with an amino acid sequence of SEQ ID NO: 2, 4, 6, 8, or 10;
vi. a nucleotide that encodes a polypeptide that is at least 85% identical to the amino acid sequence of SEQ ID NO: 2, 4, 6, 8, or 10;
wherein the screenable marker polynucleotide is operably linked to a promoter that expresses in seed, and
b) restoring male-fertility to the male-sterile plant by the complementation of the male-sterile phenotype by the one or more male-fertility restoration polynucleotides, wherein expression of the one or more male-fertility restoration polynucleotides functionally complements the male-sterility phenotype caused by the one or more mutations in the endogenous male-fertility polynucleotide in the male-sterile plant so that the male-sterile plant becomes male-fertile.

5. The method of claim 4, wherein the endogenous male-fertility polynucleotide is a Ms1, Ms5, Ms9, Ms22, Ms26, or Ms45 male-fertility polynucleotide.

6. The method of claim 4, wherein the one or more male-fertility polynucleotides is a Ms1, Ms5, Ms9, Ms22, Ms26, or Ms45 male-fertility polynucleotide.

7. The method of claim 4, wherein the promoter that expresses in seed is inserted or edited into the wheat genome so that it drives expression of the polynucleotide encoding the screenable marker.

8. The method of claim 4, wherein the promoter that expresses in seed is operably linked to a regulatory element.

9. A method of increasing seed from a wheat plant having female and male gametes, the method comprising:
self-fertilizing the wheat plant comprising (a) one or more homozygous mutations in a male-fertility polynucleotide, which results in male sterility in the wheat plant, and (b) one or more male-fertility restoration polynucleotides that functionally complements the male-sterility phenotype in the male-sterile wheat plant, wherein the one or more male-fertility polynucleotides is operably linked to a polynucleotide encoding a screenable marker for seed selection, wherein the polynucleotide is selected from the group consisting of:
i. a nucleotide sequence of SEQ ID NO:1, 3, 5, 7, or 9;
ii. a nucleotide sequence that is at least 85% identical to the nucleotide sequence of SEQ ID NO: 1, 3, 5, 7, or 9;
iii. a nucleotide fragment of the nucleotide sequence of part a;
iv. a nucleotide fragment of the nucleotide sequence of part b;
v. a nucleotide that encodes a polypeptide with an amino acid sequence of SEQ ID NO: 2, 4, 6, 8, or 10;
vi. a nucleotide that encodes a polypeptide that is at least 85% identical to the amino acid sequence of SEQ ID NO: 2, 4, 6, 8, or 10;
wherein the polynucleotide encoding a screenable marker for seed selection is operably linked to a promoter that expresses in seed; and producing wheat seed.

10. The method of claim 9, wherein the promoter that expresses in seed is inserted or edited into the wheat genome so that it drives expression of the polynucleotide encoding the screenable marker.

11. The method of claim 9, wherein the promoter that expresses in seed is operably linked to a regulatory element.

12. The method of claim 9, the method further comprising sorting the mixture of seeds into separate populations of seeds based on the expression of the screenable marker in seed, wherein the absence of the expression of the screenable marker in seed indicates the seed will produce male-sterile female plants and wherein the presence of the expression of the screenable marker in seed indicates the seed will produce male-fertile plants.

13. The method of claim 12, further comprising: selecting wheat seed that does not comprise the screenable marker expressed in the seed; growing the wheat seed into a male-sterile female wheat plant; and crossing the male-sterile female wheat plant with a cross-compatible plant to produce hybrid wheat seed.

14. The method of claim 12, further comprising selecting wheat seed that comprises the one or more male-fertility restoration polynucleotides as indicated by the presence of the expression of the screenable marker.

15. The method of claim 9, wherein the polynucleotide encoding the screenable marker is operably linked to one or more male-fertility restoration polynucleotides and not separated by a centromere.

16. The method of claim 9, wherein the endogenous male-fertility polynucleotide is a Ms1, Ms5, Ms9, Ms22, Ms26, or Ms45 male-fertility polynucleotide.

17. The method of claim 9, wherein the one or more male-fertility restoration polynucleotides is Ms1, Ms5, Ms9, Ms22, Ms26, or Ms45.

18. The method of claim 4 or 9, wherein the one or more male-fertility restoration polynucleotides has been inserted in, edited, replaced, or repositioned to be linked to the polynucleotide encoding the screenable marker using gene editing technology, chromosomal rearrangement, or combinations thereof.

19. The method of claim 4 or 9, wherein the polynucleotide encoding the screenable marker has been inserted in, edited, replaced, or repositioned to be linked to the one or more male-fertility restoration polynucleotides using gene editing technology, chromosomal rearrangement, or combinations thereof.

20. The method of claim 4 or 9, wherein the one or more male-fertility restoration polynucleotides resides on a chromosomal component from wheat, barley, oat, wheatgrass, or rye plant or a related species thereof.
